(19) United States
(12) Patent Application Publication
(10) Pub. No.: US 2025/0127841 A1
(43) Pub. Date: Apr. 24, 2025

(54) AGENT FOR IMPROVING INTESTINAL BACTERIA

(71) Applicants: FUJI OIL HOLDINGS INC., Osaka (JP); ISHIKAWA PREFECTURAL PUBLIC UNIVERSITY CORPORATION, Ishikawa (JP)

(72) Inventors: Shin KURIHARA, Kinokawa-shi, Wakayama (JP); Ichiko HIRAKAKIUCHI, Izumisano-shi, Osaka (JP); Tsutomu SAITO, Izumisano-shi, Osaka (JP); Mitsuru KATASE, Izumisano-shi, Osaka (JP)

(73) Assignees: FUJI OIL HOLDINGS INC., Osaka (JP); ISHIKAWA PREFECTURAL PUBLIC UNIVERSITY CORPORATION, Ishikawa (JP)

(21) Appl. No.: 18/834,398

(22) PCT Filed: Jan. 31, 2023
(86) PCT No.: PCT/JP2023/003031

§ 371 Date: Jul. 30, 2024

(30) Foreign Application Priority Data
Feb. 1, 2022 (JP) 2022-014261

(51) Int. Cl. A61K 38/01; A61K 36/48; A61P 1/00; A61P 31/04

(52) U.S. Cl. CPC A61K 38/011; A61K 36/48; A61P 1/00; A61P 31/04

(57) ABSTRACT

Provided is an agent for improving intestinal bacteria that contains a proteolytic enzyme-decomposed residue of a soybean protein material and inhibits the growth of opportunistic bacteria.

### TECHNICAL FIELD

### Related Application

This application claims the benefit of priority of Japanese Patent Application No. 2022-014261 filed with the Japan Patent Office on Feb. 1, 2022. The basic priority application is incorporated herein by reference in its entirety.

The present invention relates to an agent for improving intestinal bacteria containing a proteolytic enzyme-decomposed residue of a soybean protein material.

### Background Art

Many kinds and a large number of intestinal bacteria live in the human intestines, and the intestinal bacteria are roughly divided into three groups (good bacteria, bad bacteria, and opportunistic bacteria). These bacteria are closely related to each other and are in a complex balance. Among the intestinal bacteria, the most abundant bacteria are opportunistic bacteria, the second most abundant bacteria are good bacteria, and the least abundant bacteria are bad bacteria.

Opportunistic bacteria whose function is unknown and which do not cause a short-term disease account for 90% of human intestinal bacteria (Non-Patent Literature 1). Among opportunistic bacteria, some strains of the genus Bacteroides cause adverse effects under certain conditions. For example, there are Bacteroides caccae (Non-Patent Literature 2) which eats mucin and increases sensitivity to pathogenic bacteria, Bacteroides dorei (Non-Patent Literature 3) which secrets lipopolysaccharide inhibiting immune maturation in infancy, and Bacteroides fragilis (Non-Patent Literature 4) which may promote the development of colorectal cancer in familial adenomatous polyposis patients when coexisting with E. coli in the colon.

To prevent such adverse effects caused by opportunistic bacteria and to promote growth of good bacteria to maintain a healthy intestinal environment, it is required to inhibit excessive growth of opportunistic bacteria.

As an agent for improving intestinal bacteria that inhibits growth of bacteria belonging to the genus Bacteroides, which are opportunistic bacteria, and can be used for food and drink products, an intestinal bacteria activator that inhibits growth of bacteria belonging to the genus Bacteroides (Patent Document 1), a composition containing a preparation of a plant belonging to the genus Perilla that inhibits growth of bacteria belonging to the genus Bacteroides and the genus Clostridium and has a prebiotic effect (Patent Document 2), and an antimicrobial agent containing an endolysin mutant that has LPS-destroying activity and destroys cell walls of gram-negative bacteria belonging to the family Bacteroides (Patent Document 3) have been disclosed.

However, there is still a need for a new agent for improving intestinal bacteria that inhibits growth of bacteria belonging to the genus Bacteroides.

### CITATION LIST

### Patent Document

Patent Document 1: JP 2007-330177 A
Patent Document 2: JP 2016-525520 T
Patent Document 3: JP 2012-500642 T

### Non-Patent Literatures

Non-Patent Literature 1: Qin et al., NATURE, Vol. 464(4) March 2010, 59-65, and Supplementary Information
Non-Patent Literature 2: Desai et al., Cell, 167, 1339-1353, Nov. 17, 2016
Non-Patent Literature 3: Vatanen et al., Cell 165, 842-853, May 5, 2016
Non-Patent Literature 4: Dejea et al., Science, 2018 Feb. 2, 359(6375), 592-597

### SUMMARY OF INVENTION

### Technical Problem

An object of the present invention is to provide an agent for improving intestinal bacteria that inhibits growth of bacteria belonging to the genus Bacteroides, which is one of opportunistic bacteria.

### Solution to Problem

As a result of intensive studies on the above problems, the present inventors have found that the growth of bacteria belonging to the genus Bacteroides can be inhibited by using an enzyme-decomposed residue which is an insoluble fraction obtained by treating soybean protein with a proteolytic enzyme in an aqueous system and separating the treated product, and thus completed the present invention.

That is, the present invention relates to
(1) an agent for improving intestinal bacteria containing a proteolytic enzyme-decomposed residue of a soybean protein material;
(2) the agent for improving intestinal bacteria according to (1), wherein the proteolytic enzyme-decomposed residue has a molecular weight distribution in which an area ratio of less than 200 Da is from 0 to 20%, an area ratio of 2000 Da or more and less than 30000 Da is from 40 to 80%, and an area ratio of 30000 Da or more is from 10 to 40% in a measurement result of the molecular weight distribution; and
(3) the agent for improving intestinal bacteria according to (1) or (2), which inhibits growth of bacteria belonging to the genus Bacteroides.

### Advantageous Effects of Invention

The agent for improving intestinal bacteria of the present invention can inhibit the growth of bacteria belonging to the genus Bacteroides. More specifically, the agent for improving intestinal bacteria of the present invention can specifically inhibit the growth of bacteria belonging to the genus Bacteroides. Still more specifically, the agent for improving intestinal bacteria of the present invention can specifically inhibit the growth of bacteria belonging to the genus Bacteroides and promote growth of some lactic acid bacteria and bifidobacteria.

### BRIEF DESCRIPTION OF DRAWINGS

FIG. 1 is a graph showing results of Example 4.

### DESCRIPTION OF EMBODIMENTS

In one aspect, the present invention provides an agent for improving intestinal bacteria containing a proteolytic enzyme-decomposed residue of a soybean protein material.

The agent for improving intestinal bacteria of the present aspect uses a soybean protein material as a raw material. The type of the soybean protein material used is not particularly limited, and examples thereof include whole soybean, defatted soybean, isolated soybean protein, concentrated soybean protein, fractionated soybean protein, soybean milk, defatted soybean milk, and various processed products thereof. In one embodiment, the soybean protein material has a protein content of 50 mass % or more, preferably 70 mass % or more, more preferably 80 mass % or more, and even more preferably 90 mass % or more in terms of a dry weight. In one embodiment, the soybean protein material is isolated soybean protein.

The agent for improving intestinal bacteria of the present aspect contains an enzyme-decomposed residue (also referred to as an insoluble fraction or a precipitated fraction) obtained by treating a soybean protein material with a proteolytic enzyme in an aqueous system and then separating and collecting a water-soluble fraction. Such a decomposed residue is a relatively high molecular weight fraction, and thus also referred to as IMF.

The proteolytic enzyme (protease) used in the above treatment can be appropriately selected from proteases classified into “metal protease”, “acid protease”, “thiol protease”, and “serine protease” in the classification of proteases, preferably proteases classified into “metal protease”, “thiol protease”, and “serine protease”, regardless of whether it originates from animals, plants, or microorganisms.

This classification of proteases is a classification method based on the type of amino acid at the active center, which is usually performed in the field of enzyme science. Representative examples of the “metal protease” include Bacillus neutral protease, Streptomyces neutral protease, Aspergillus neutral protease, and “Thermoase”, representative examples of the “acid protease” include pepsin, Aspergillus acid protease, and “Sumitum AP”, representative examples of the “thiol protease” include bromelain and papain, and representative examples of the “serine protease” include trypsin, chymotrypsin, subtilisin, Streptomyces alkaline protease, “Alcalase”, and “Bioplase”.

The reaction pH and the reaction temperature of the treatment with the proteolytic enzyme only need be set in accordance with the characteristics of the enzyme used, and the reaction only need be usually carried out at a reaction pH near the optimum pH and at a reaction temperature near the optimum temperature. The reaction can be generally carried out at a pH in a range of 2 to 8. The reaction can be generally carried out at a reaction temperature of from 20 to 80° C., and preferably of from 40 to 60′C. After the reaction, the reaction product is heated at a temperature sufficient to deactivate the enzyme (about 60 to 170° C.) to deactivate the remaining enzyme activity.

After the enzyme treatment, a water-soluble fraction and a decomposed residue are separated, and the decomposed residue is collected. The separation method is not particularly limited, and a separation method such as centrifugation or filtration can be used. Examples of the conditions of centrifugation include conditions of from 1000 to 5000 rpm for from 5 to 30 minutes. Examples of the filtration include microfiltration and filter press. The decomposed residue may be purified by washing with water once or a plurality of times. In one embodiment, the obtained decomposed residue may be further subjected to enzyme treatment to separate a water-soluble fraction and the decomposed residue and collect the decomposed residue. In a further embodiment, the above operation may be further performed a plurality of times.

The obtained decomposed residue may be used as the agent for improving intestinal bacteria of the present aspect as it is or after neutralization and sterilization treatment as necessary. In one embodiment, the obtained decomposed residue can be dried and used as a powder. The drying method is not particularly limited, and examples thereof include spray drying, fluidized bed drying, and freeze drying.

In one embodiment, in a case where the molecular weight of the decomposed residue or the agent for improving intestinal bacteria is measured by a method described in the following “Molecular weight distribution”, the area ratio of less than 2000 Da is from 0 to 20%, for example from 1 to 15% or from 2 to 10%; the area ratio of 2000 Da or more and less than 30000 Da is from 40 to 80%, for example from 45 to 75% or from 50 to 70%; and the area ratio of 30000 Da or more is from 10 to 40%, for example from 15 to 30% or from 20 to 25%, in the molecular weight distribution.

In one embodiment, in the decomposed residue or the agent for improving intestinal bacteria, the proportion of hydrophobic amino acids (Tyr, Phe, Val, Ile, Leu, and Trp) in the amino acid composition is 30 mass % or more, for example, 35 mass % or more.

The agent for improving intestinal bacteria of the present aspect can inhibit the growth of bacteria belonging to the genus Bacteroides. In a more specific embodiment, the agent for improving intestinal bacteria of the present aspect can specifically inhibit the growth of bacteria belonging to the genus Bacteroides. In an even more specific embodiment, the agent for improving intestinal bacteria of the present aspect can specifically inhibit the growth of bacteria belonging to the genus Bacteroides and promote the growth of some lactic acid bacteria and bifidobacteria, for example, Lactobacillus gasseri. Lactobacillus johnsonii, Lactobacillus plantarum, Lactobacillus reuteri, Bifidobacterium bifidum, Bifidobacterium breve, Bifidobacterium catenulatum, Bifidobacterium longum, Bifidobacterium pseudocatenulatum, and Bifidobacterium pseudolongum.

In the present specification, “inhibiting the growth of bacteria” means that, for example, a proportion of a target bacterium to a control determined by the method described in “Evaluation of Promotive and Inhibitive Actions of Growth of Intestinal Bacteria” described below is less than 100%, for example, less than 90%, less than 80%, or less than 70%. In the present specification, “promoting the growth of bacteria” means that, for example, the proportion is 100% or more, for example, 110% or more, 120% or more, or 130% or more.

In one embodiment, the agent for improving intestinal bacteria of the present aspect may be a pharmaceutical composition. The above-described agent for improving intestinal bacteria may be formulated in accordance with a common method (see, for example, Remington's Pharmaceutical Science, latest edition. Mark Publishing Company, Easton. U.S.A.), and may contain a pharmaceutically acceptable carrier or additive. Examples thereof include, but are not limited to, a surfactant, an excipient, a colorant, a flavoring agent, a preservative, a stabilizer, a buffer, a suspending agent, an isotonic agent, a binder, a disintegrating agent, a lubricant, a fluidity promoter, and a taste masking agent, and furthermore, a commonly used carrier can be appropriately used. Specific examples thereof include light silicic anhydride, lactose, crystalline cellulose, mannitol, starch, carmellose calcium, carmellose sodium, hydroxypropylcellulose, hydroxypropylmethylcellulose, polyvinylacetal diethylaminoacetate, polyvinylpyrrolidone, gelatin, medium-chain fatty acid triglyceride, polyoxyethylene hydrogenated castor oil 60, white sugar, carboxymethylcellulose, corn starch, and inorganic salts.

The method for administering the pharmaceutical composition is not particularly limited, and is preferably oral administration. The form of the pharmaceutical composition is not particularly limited, and examples thereof include a concentrated liquid, a powder, a granule, a pill, a tablet, a capsule, and a drink.

Examples of a subject of administration of the pharmaceutical composition include, but are not limited to, humans, animals including humans, and animals not including humans, such as humans, mice, rats, monkeys, pigs, cattle, horses, goats, sheep, donkeys, does, cats, rabbits, hamsters, guinea pigs, chickens, other domestic animals, poultry, and pets.

The pharmaceutical composition may be administered three times a day, twice a day, or once a day. For example, the pharmaceutical composition may be administered to the subject at, or before or after each meal, or the pharmaceutical composition may be administered to the subject once a day, for example, at, or before or after breakfast, at, or before or after lunch, or at, or before or after dinner. Alternatively, the pharmaceutical composition may be administered between meals.

The dosage amount of the pharmaceutical composition can be appropriately adjusted and is not particularly limited. Examples of the dosage amount include 5 mg to 20 g, 10 mg to 10 g, 20 mg to 5 g, 40 mg to 1 g, and 50 to 500 mg as the agent for improving intestinal bacteria per day for an adult in terms of solid content.

In one embodiment, the agent for improving intestinal bacteria of the present aspect may be a food composition. The food composition may contain the carriers and additives mentioned above for the pharmaceutical composition. The form of the food composition is not particularly limited, and examples thereof include a concentrated liquid, a powder, a granule, a tablet, a pill, a gum, a candy, a capsule, a paste, a jelly, and a drink. The food composition may be used as it is or in a state of a concentrated liquid, a powder, a granule, a paste, or the like to prepare a food product, for example, by being mixed and cooked with another raw material, or by being added to another food product. The food composition may be used as a food additive as it is or in the state of a concentrated liquid, a powder, a granule, a paste, or the like, to which another raw material is added as necessary.

Examples of the other raw material that can be included in the food composition or food product include: seasonings such as sugar, salt, soy sauce, miso (bean paste), and vinegar; sweeteners such as honey, maple syrup, oligosaccharides, and high-intensity sweeteners; acidulants such as citric acid, malic acid, tartaric acid, and acetic acid; bittering agents such as caffeine and naringin; spices such as oregano, pepper, cinnamon, ginger, turmeric, chili pepper, basil, paprika, and Japanese horse-radish; oils such as salad oil, sesame oil, safflower oil, sunflower oil, rapeseed oil, fish oil, beef tallow, and lard; flavor oils; extracts such as yeast extract, seafood extract, beef extract, pork extract, and chicken extract; fruit juices; vegetable juices; vitamins; minerals; sugars; oligosaccharides; polysaccharides; dietary fibers; amino acids; peptides; fatty acids; coenzymes; flavors; emulsifiers; thickeners; and color formers.

The food composition or food product may be taken three times a day, twice a day, or once a day. In one embodiment, the food composition or food product may be taken as a functional food product, for example, at, or before or after each meal, or may be taken once a day, for example, at, or before or after breakfast, at, or before or after lunch, or at, or before or after dinner. Alternatively, it may be taken between meals.

The intake amount of the food composition or food product can be appropriately adjusted depending on the product design, and is not particularly limited. Preferable examples of the intake amount include from 5 mg to 20 g, from 10 mg to 10 g, from 20 mg to 5 g. from 40 mg to 1 g, and from 50 to 500 mg as the agent for improving intestinal bacteria per day for an adult.

In one aspect, the present invention provides a method for producing the above-described agent for improving intestinal bacteria, pharmaceutical composition, food composition, or food product. The raw materials used in the method of the present aspect, the conditions in the method of the present aspect, and the like are as described above.

In one aspect, the present invention provides use of a proteolytic enzyme-decomposed residue of a soybean protein material for producing an agent for improving intestinal bacteria, or a pharmaceutical composition or a food composition for improving intestinal bacteria. The raw materials, conditions, and the like in the use of the present aspect are as described above.

In one aspect, the present invention provides a method for improving intestinal bacteria by administering the agent for improving intestinal bacteria, the pharmaceutical composition, the food composition, or the food product to a subject, or causing a subject to take the agent for improving intestinal bacteria, the pharmaceutical composition, the food composition, or the food product. In a more specific embodiment, the method for improving intestinal bacteria is a method for inhibiting the growth of bacteria belonging to the genus Bacteroides in the intestine. The form, the subject of administration or intake, the administration or intake amount, and the like in the method of the present aspect are as described above.

### Measurement Methods

In the present specification, components and physical properties are measured in accordance with the following methods.

### Protein Content

The protein content is measured by the Kjeldahl method. Specifically, a mass of nitrogen measured by the Kjeldahl method relative to a mass of a protein material dried at 105° C., for 12 hours is represented by “mass %” as a protein content in the dried product. The nitrogen conversion coefficient is 6.25. Basically, the value is calculated by rounding off to the first decimal place.

### Molecular Weight Distribution

A sample solution is prepared by adjusting a concentration of protein in a sample to 0.1 mass % with an eluent, and then filtering the sample with a 0.2 μm filter. A gel filtration system is assembled by connecting two types of columns in series, known proteins and the like (Table 1) used as molecular weight markers are first charged, and a calibration curve is determined from the relationship between the molecular weight and the retention time. The sample solution is then charged, and the content ratio % of each molecular weight fraction is determined by the proportion of the area of a specific molecular weight range (time range) to the area of the total absorbance chart (1st column is “TSK gel G3000SW_{XL}” (SIGMA-ALDRICH) and 2nd column “TSK gel G2000SW_{XL}” (SIGMA-ALDRICH); an eluent is a mixture of 1% SDS, 1.17% NaCl, and a 50 mM phosphate buffer (pH7.0); 23° C.; a flow rate of 0.4 mL/min; detection at UV 220 nm). Basically, the value is calculated by rounding off to the first decimal place.

TABLE 1
Molecular weight markers

| Marker | Molecular weight |
|---|---|
| Thyroglobulin | 335,000 |
| γ-globulin | 150,000 |
| Albumin | 67,000 |
| Peroxidase | 43,000 |
| Myoglobin | 18,000 |
| Cytochrome C | 12,384 |
| Insulin | 5,734 |
| Glutathione | 307 |
| p-Aminobenzoate | 137 |

### Evaluation of Promotive and Inhibitive Actions of Growth of Intestinal Bacteria

To evaluate the influence of oral administration of the agent for improving intestinal bacteria on the growth of intestinal bacteria, a sample is artificially digested by the following method, and the growth of target intestinal bacteria is compared between a medium to which the artificially digested product is added and a medium to which the artificially digested product is not added. The specific method is as follows.

### (1) Artificial Digestion of Sample

A sample is artificially digested in accordance with the method described in Kimikazu Iwami et al., “Molecular Recognition and Inclusion Molecular Modeling of Bile Acids by Soy Protein”, Soy Protein Research Vol. 4 (2001), 58-64. Specifically, the sample is suspended in diluted hydrochloric acid to prepare a 5% sample suspension of pH2. While the suspension is kept at 37° C. pepsin is added thereto in an amount of 2% based on the substrate to perform reaction for 4 hours, then the mixture is adjusted to pH8 with 2M sodium hydroxide and pancreatin is added thereto in an amount of 1% based on the substrate to perform enzymatic reaction for 24 hours. The precipitated fraction is collected by centrifugation and suspended in 0.1M sodium chloride aqueous solution, and the obtained suspension is adjusted to pH4.5 with 0.5M hydrochloride and centrifuged to obtain a precipitate. This step is repeated again, and the resulting precipitate is freeze-dried to obtain an artificially digested product of the sample.

### (2) Evaluation of Promotive and Inhibitive Actions of Growth of Intestinal Bacteria

The simultaneous culture assay described in JP 2018-108968 A is modified for evaluation.

### (I) Preparation of Medium

(i-i) Preparation of Preculture Medium and Negative Control Test Medium (Gifu Anaerobic Medium (GAM))

GAM bouillon (available from Nissui Corporation) and demineralized water are mixed and dissolved thoroughly with a stirrer, and then demineralized water is added such that the concentration of the GAM bouillon is 5.9 (w/v) %. The obtained mixed solution is charged into a heat-resistant bottle and autoclaved at 115° C. for 15 minutes. With a lid half-opened, the bottle is quickly placed in an Anaeropack square jar (available from Mitsubishi Gas Chemical Company Inc.) under anaerobic conditions and allowed to stand overnight to prepare GAM for preculture and negative control test.

(i-ii)

The prepared GAM is dispensed into a 96-deep well plate in an amount of 500 μL, each with an 8-channel multi-pipette, and the 96-deep well plate is sealed with a gas-permeable seal for a microplate, placed in an Anaeropack square jar under anaerobic conditions, and allowed to stand overnight.

(i-iii) Preparation of Medium Supplemented with Artificially Digested Product of Agent for Improving Intestinal Bacteria

The artificially digested product obtained in (1) is mixed with demineralized water to have a concentration of 5%, and dissolved thoroughly with a stirrer. The mixed solution is charged into a heat-resistant bottle and autoclaved (121° C., 20 minutes). With a lid half-opened, the bottle is quickly placed in an Anaeropack square jar (available from Mitsubishi Gas Chemical Company Inc.) under anaerobic conditions and allowed to stand overnight to prepare a 5% (w/v) artificially digested product solution. GAM is prepared by the same procedure as (i-i) to have a concentration of 10/9 times 5.9 (w/v) %.

In an anaerobic chamber (available from Baker Ruskinn, InvivO2), 5 mL of the 5% artificially digested product solution is added to 45 mL of the GAM (under a nitrogen gas atmosphere, oxygen of 1% or less) to prepare a medium supplemented with 0.5 (w/v) % artificially digested product.

(ii) Selective Growth inhibition or Promotion Evaluation Test using Various Intestinal Bacteria

In an anaerobic chamber, the plate for preculture prepared in (i-ii) is inoculated with various target bacterial cells, which are glycerol stock, with a sterilized toothpick, and preculture is performed at 37° C. under anaerobic conditions in an Anaeropack square jar.

The medium supplemented with artificially digested product prepared in (i-iii) or the GAM prepared in (i-i) for negative control segment test is dispensed into a 96-deep well plate in an amount of 500 μL each with an 8-channel multi-pipette, and the 96-deep well plate is sealed with a gas-permeable seal for a microplate, placed in an Anaeropack square jar under anaerobic conditions, and allowed to stand overnight.

A pin of a dry-heat-sterilized copy plate 96 (Tokken TK-CP96) is inserted to the bottom of the preculture plate and then inserted to the bottom of the 96-deep well plate containing the medium to perform inoculation of each bacterial cell. A gas-permeable seal for a microplate is affixed to the 96-deep well plate and culturing is performed under the anaerobic conditions in the Anaeropack square jar. After culturing for 24 hours, absorbance (turbidity) at 600 nm is measured with a microplate reader (available from Thermo Scientific). After suspending the bacterial cells by pipetting the culture solution with an 8-channel multi-pipette, 20 μL of the suspension is added to 180 μL of phosphate buffered saline charged into a 96-well titer plate, and mixed by pipetting with an 8-channel multi-pipette. Blank measurement is performed using the GAM prepared in (i-i). Next, from the obtained measured values, the growth promotion factor (=measured value of artificially digested product−added segment/measured value of negative control) is calculated for each bacterial cell.

### EXAMPLES

Hereinafter, embodiments of the present invention will be more specifically described by examples and the like. Note that unless otherwise specified, “%”, “part”, and the like in the examples are on mass basis.

### Example 1: Preparation of Agent for Improving Intestinal Bacteria

100 parts by weight of soybean protein isolate (Fujipro F) was dissolved in water of pH7 to have a concentration of 10%, and Protein AY-10 (derived from Bacillus licheniformis, available from Amano Enzyme Inc.) was added thereto in an amount of 2% based on a substrate, followed by reaction at 50° C., for 4 hours. The reaction product was heated at 80° C., for 15 minutes to inactivate the enzyme and centrifuged (3000 rpm, 20 minutes) to obtain a precipitate. The obtained precipitate was freeze-dried to obtain a powdery agent for improving intestinal bacteria of the present invention (Sample A).

### Example 2: Measurement of Molecular Weight Distribution of Agent for Improving Intestinal Bacteria

Molecular weight distributions of Sample A and a precipitated fraction (Sample B) obtained by preparing Sample A in accordance with the artificial digestion method described in the above “Evaluation of Promotive and Inhibitive Actions of Intestinal Bacteria” were measured in accordance with the method described in the above “Molecular Weight Distribution”. The results are presented in Table 2.

TABLE 2
Unit (%)

| | Sample A | Sample B |
|---|---|---|
| 30000 Da or more | 17.46 | 27.1 |
| 30000 to 2000 Da | 66.96 | 66.37 |
| Less than 2000 Da | 18.2 | 10.15 |

### Example 3: Measurement of Amount of Hydrophobic Amino Acids in Agent for Improving Intestinal Bacteria

The proportion of hydrophobic amino acids (Tyr, Phe, Val, Ile, Leu, and Trp) in the amino acid composition was measured and found to be 34% in sample A and 45% in sample B.

Note that as shown in Examples 2 and 3, the molecular weight distribution and the proportion of hydrophobic amino acids are substantially maintained even after artificial digestion, and it is expected that the effect is maintained.

### Example 4: Confirmation of Effect of Growth Inhibition or Promotion of Agent for Improving Intestinal Bacteria for Various Intestinal Bacteria

Sample A was subjected to measurement in accordance with the method described in the above “Evaluation of Promotive and Inhibitive Actions of Growth of Intestinal Bacteria”.

The bacterial species used in the present test are as follows.
25 strains of intestinal resident bacteria including 12 most dominant strains of human intestinal resident bacteria

### Most Dominant Species of Human Intestinal Resident Bacteria

Bacteroides caccae
Bacteroides dorei
Bacteroides finegoldii
Bacteroides fragilis
Bacteroides intestinalis
Bacteroides ovatus
Bacteroides stercoris
Bacteroides thetaiotaomicron
Bacteroides uniformis
Bacteroides vulgatus
Blautia hansenii
Clostridium nexile
Eubacterium ventriosum

### Lactic Acid Bacteria

Lactobacillus gasseri
Lactobacillus johnsonii
Lactobacillus plantarum
Lactobacillus reuteri

### Bifidobacteria

Bifidobacterium bifidum
Bifidobacterium breve
Bifidobacterium catenulatum
Bifidobacterium longum
Bifidobacterium pseudocatenulatum
Bifidobacterium pseudolongum

### Butyric Acid-Producing Bacteria

Clostridium indolis
Clostridium ramosum

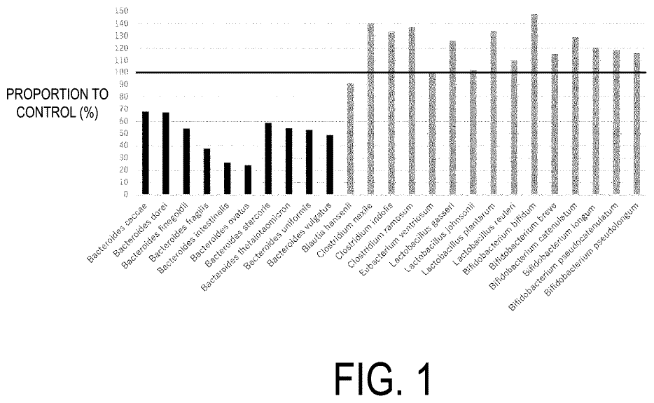

The results are shown in FIG. 1. As shown in FIG. 1, the agent for improving intestinal bacteria of the present invention specifically inhibited the growth of bacteria belonging to the genus Bacteroides. In addition, the growth of lactic acid bacteria and bifidobacteria was promoted.

### INDUSTRIAL APPLICABILITY

According to the present invention, it is possible to provide an agent for improving intestinal bacteria capable of inhibiting the growth of bacteria belonging to the genus Bacteroides. The agent for improving intestinal bacteria can be used for a pharmaceutical composition, a food composition, a food product, or the like.

1. An agent for improving intestinal bacteria, comprising a proteolytic enzyme-decomposed residue of a soybean protein material.

2. The agent for improving intestinal bacteria according to claim 1, wherein the proteolytic enzyme-decomposed residue has a molecular weight distribution in which an area ratio of less than 2000 Da is from 0 to 20%, an area ratio of 2000 Da or more and less than 30000 Da is from 40 to 80%, and an area ratio of 30000 Da or more is from 10 to 40% in a measurement result of a molecular weight distribution.

3. The agent for improving intestinal bacteria according to claim 1, which inhibits growth of bacteria belonging to the genus Bacteroides.

4. A method for improving intestinal bacteria, comprising administering to a subject in need thereof a proteolytic enzyme-decomposed residue of a soybean protein material.

5. The method for improving intestinal bacteria according to claim 4, wherein the proteolytic enzyme-decomposed residue has a molecular weight distribution in which an area ratio of less than 2000 Da is from 0 to 20%, an area ratio of 2000 Da or more and less than 30000 Da is from 40 to 80%, and an area ratio of 30000 Da or more is from 10 to 40% in a measurement result of a molecular weight distribution.

6. The method for improving intestinal bacteria according to claim 4, wherein the proteolytic enzyme-decomposed residue of a soybean protein material inhibits growth of bacteria belonging to the genus Bacteroides.
